(19) United States
(12) Patent Application Publication
(10) Pub. No.: US 2012/0277410 A1
(43) Pub. Date: Nov. 1, 2012

(54) DEHYDRIN-BASED ENTROPIC BRISTLE DOMAIN SEQUENCES AND THEIR USE IN RECOMBINANT PROTEIN PRODUCTION

(75) Inventors: Alan Keith Dunker, Indianapolis, IN (US); Aaron Andrew Santner, Avon, IN (US); Vladimir N. Uversky, Tampa, FL (US)

(73) Assignee: MOLECULAR KINETICS INCORPORATED, Indianapolis, IN (US)

(21) Appl. No.: 13/435,989

(22) Filed: Mar. 30, 2012

Related U.S. Application Data
(60) Provisional application No. 61/469,481, filed on Mar. 30, 2011.

(51) Int. Cl. C07K 19/00; C12N 5/10; C12N 1/21; C12P 21/02; C12N 15/62; C12N 15/63

(52) U.S. Cl. 530/370; 536/23.4; 435/320.1; 435/252.33; 435/69.7; 435/419; 435/348; 435/325; 435/358; 435/365; 435/367; 435/350; 435/369

(57) ABSTRACT

Fusion polypeptides, polynucleotides encoding fusion polypeptides, expression vectors, kits, and related compositions and methods for recombinant protein production are provided, wherein the fusion polypeptides comprise a sequence derived from a plant dehydrin protein covalently linked to a heterologous protein sequence in order to enhance the solubility and folding of the heterologous protein sequence and to reduce its aggregation.

### CROSS-REFERENCE TO RELATED APPLICATION

This application claims the benefit under 35 U.S.C. §119(e) of U.S. Provisional Patent Application No. 61/469,481, filed Mar. 30, 2011, which application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 670098_{—}408_SEQUENCE_LISTING.txt. The text file is 9 KB, was created on Mar. 30, 2012, and is being submitted electronically via EFS-Web.

### FIELD OF THE INVENTION

The present invention relates generally to compositions and methods for recombinant protein production and, more particularly, to fusion polypeptides, polynucleotides encoding fusion polypeptides, expression vectors, kits, and related methods for recombinant protein production.

### DETAILED OF THE RELATED ART

A large percentage of the proteins identified via the different genome sequencing efforts have been difficult to express and/or purify as recombinant proteins using standard methods. For example, a trial study using Methanobacterium thermoautotrophicum as a model system identified a number of problems associated with high throughput structure determination (Christendat, D. et al. (2000). Prog Biophys Mol Biol 73, 339-45; Christendat et al. (2000). Nat Struct Biol 7, 903-9). The complete list of genome-encoded proteins was filtered to remove proteins with predicted transmembrane regions or homologues to known structures. When these filtered proteins were taken through the cloning, expression, and structural determination steps of a high throughput process, only about 50% of the selected proteins could be purified in a state suitable for structural studies, with roughly 45% of large expressed proteins and 30% of small expressed proteins failing due to insolubility. The study concluded that considerable effort must be invested in improving the attrition rate due to proteins with poor expression levels and/or unfavorable biophysical properties (ibid).

Similar results have been observed for other prokaryotic proteomes. One study reported the successful cloning and attempted expression of 1376 (73%) of the predicted 1877 genes of the Thermotoga maritima proteome. However, crystallization conditions were able to be determined for only 432 proteins (23%). A significant component of the decrease between the cloned and crystallized success levels was due to poor protein solubility and stability (Kuhn et al. (2002). Proteins 49, 142-5).

Similarly low success rates have been reported for eukaryotic proteomes. A study of a sample set of human proteins, for example, reported that the failure rate using high-throughput methods for three classes of proteins based on cellular location was 50% for soluble proteins, 70% for extracellular proteins, and more than 80% for membrane proteins (Braun, P. et al. (2002). Proc Natl Acad Sci USA 99, 2654-9).

Interactions between individual recombinant proteins are responsible for a significant number of the previously mentioned failures. In a high-throughput structural determination study, Christendat and colleagues found that 24 of 32 proteins that were classified by nuclear magnetic resonance as aggregated displayed circular dichroism spectra consistent with stable folded proteins, suggesting that these proteins were folded properly but aggregated due to surface interactions (Christendat et al. (2000) Prog. Biophys. Mol. Biol. supra). One possible explanation for this is that these proteins function in vivo as part of multimeric units but when they are recombinantly expressed, dimerization domains are exposed that mediate protein-protein interactions.

Prior methods used to increase recombinant protein stability include production in E. coli strains that are deficient in proteases and production of fusions of bacterial protein fragments to a recombinant polypeptide/protein of interest (Gottesman, S. & Zipser, D. (1978). J Bacteriol 133, 844-51; Itakura, K. et al. (1977). Science 198, 1056-63). In addition, fusing a leader sequence to a recombinant protein may cause a gene product to accumulate in the periplasm or be excreted, which may result in increased recovery of properly folded soluble protein (Nilsson, B. et al. (1985). EMBO J 4, 1075-80; Abrahmsen, L. et al. (1986). Nucleic Acids Res 14, 7487-500). These strategies have advantages for some proteins but they generally do not succeed when used, for example, with membrane proteins or proteins capable of strong protein-protein interactions.

Fusion polypeptides have also been used as an approach for improving the solubility and folding of recombinant polypeptides/proteins produced in E. coli (Zhan, Y. et al. (2001). Gene 281, 1-9). Some commonly used fusion partners which have been linked to heterologous protein sequences of interest include calmodulin-binding peptide (CBP), glutathione-S-transferase (GST), thioredoxin (TRX), and maltose-binding protein (MBP) (Vaillancourt, P. et al. (1997). Biotechniques 22, 451-3; Smith, D. B. (2000). Methods Enzymol 326, 254-70; Hammarstrom, M. et al. (2002). Protein Sci 11, 313-21; Sachdev, D. & Chirgwin, J. M. (2000). Methods Enzymol 326, 312-21). Glutathione-S-transferase and maltose-binding protein, for example, have been found to increase the recombinant protein purification success rate when fused to a heterologous sequence in a controlled trial of 32 human test proteins (Braun, P. et al (2002) supra). Further, maltose-binding protein domain fusions have been shown to increase the solubility of recombinant proteins (Kapust, R. B. & Waugh, D. S. (1999). Protein Science 8, 1668-74; Braun, P. et al. (2002) supra; Hammarstrom, M. et al. (2002) supra). Maltose-binding protein may further benefit recombinant protein solubility and folding in that it may have chaperone-like properties that assist in folding of the fusion partner (Richarme, G. & Caldas, T. D. (1997). J Biol Chem 272, 15607-12; Bach, H. et al (2001). J Mol Biol 312, 79-93). However, these fusion approaches used to date have not been amendable to all classes of proteins, and have thus met with only limited success.

Entropic bristles have been used in a variety of polymers to reduce aggregation of small particles such as latex particles in paints and to stabilize a wide variety of other colloidal products (Napper, D. H. (1983). (Ottewill & Rowell, eds.), pp. 18-30. Academic Press, New York). More specifically, the singular entropic bristles elements, comprised of highly flexible, non-aggregating polymer chains are often assembled to form entropic brushes. When these brushes are affixed to the surfaces of particles (e.g. latex beads) they are capable of preventing particle aggregation. This is achievable because the entropic bristle extends from the particle surface to which it is attached, and through processes of random motion about this attachment point, sweeps out a significant region of space to entropically exclude other molecules (ibid). Analogously, in protein chemistry entropic bristles domains (EBDs) have been observed (Hoh, J. H. (1998). Proteins 32, 223-8). These EBD polypeptides have a tendency to lack secondary structure, and again through random motions result in the creation a large area of three-dimensional space which excludes large molecules, but does not exclude small molecules such as water, salts, metal ions, or cofactors (ibid). Subsequent to here, the term EBD will generically describe both polymeric and protein based entropic bristles.

In addition to preventing aggregation, EBDs can also function as steric stabilizers and operate through steric hindrance stabilization (Napper, D. H. (1983) supra). Napper described characteristics that contribute to steric stabilization functions, including (1) they have an amphipathic sequence; (2) they are attached to the colloidal particle by one end rather than being totally adsorbed; (3) they are soluble in the medium used; (4) they are mutually repulsive; (5) they are thermodynamically stable; and (6) they exhibit stabilizing ability in proportion to their length. Steric stabilizers intended to function in aqueous media extend from the surface of colloidal molecules thus transforming their surfaces from hydrophobic to hydrophilic. The fact that sterically stabilized particles are thermodynamically stable leads them to spontaneously re-disperse when dried residue is reintroduced to solvent. Entropic bristles can adopt random-walk configurations in solution (Milner, S. T. (1991). Science 251, 905-14). These chains extend from an attachment point because of their affinity for the solvent. This affinity is due in part to the highly charged nature of the entropic bristle sequence.

While certain prior approaches have met with some success, there remains a need for new compositions and methods for improving the properties and characteristics of recombinant proteins, e.g., improving solubility, stability, yield and/or folding of recombinant proteins. The present invention addresses these needs and offers other related advantages by employing entropic bristle domain sequences as fusion partners in recombinant protein production, as described herein.

### SUMMARY OF THE INVENTION

According to a general aspect of the present invention, there are provided isolated fusion polypeptides comprising at least one entropic bristle domain (EBD) sequence and at least one heterologous polypeptide sequence of interest. The fusion polypeptides of the invention offer a number of advantages over prior fusion polypeptides and methods relating thereto.

More specifically, in certain embodiments, an EBD sequence used according to the present invention is, or is derived from, a plant dehydrin sequence. Accordingly, the present invention provides, in more specific embodiments, an isolated fusion polypeptide comprising a plant dehydrin EBD polypeptide sequence and a heterologous polypeptide sequence of interest.

In more specific embodiments, the plant dehydrin polypeptide sequence used in the present invention comprises a plant dehydrin ERD10 polypeptide sequence or a plant dehydrin ERD14 polypeptide sequence.

The plant dehydrin sequences used in the present invention may come from any suitable plant source. For example, in certain preferred embodiments, the sequences are derived from a plant species such as Arabidopsis or Brassica.

In certain more specific embodiments, the plant dehydrin polypeptide sequence comprises at least a fragment of an Arabidopsis thaliana ERD10 polypeptide sequence set forth in SEQ ID NO: 1, or a variant thereof, as described herein.

In other embodiments, the plant dehydrin polypeptide sequence comprises at least a fragment of a Brassica napus dehydrin ERD10 polypeptide sequence set forth in SEQ ID NO: 5, or variant thereof, as described herein.

In still other embodiments, the plant dehydrin polypeptide sequence comprises at least a fragment of an A. thaliana dehydrin ERD14 polypeptide sequence set forth in SEQ ID NO: 3, or a variant thereof, as described herein.

Preferably, by using such plant dehydrin sequences in fusion polypeptides and other embodiments of the invention, a fusion polypeptide will have increased solubility, reduced aggregation and/or improved folding relative to the heterologous polypeptide sequence in the absence of the dehydrin sequences.

In still another embodiment, the EBD sequence of a fusion polypeptide of the invention comprises a combination of any one or more of the EBD sequences set forth herein.

According to another embodiment of the invention, an EBD sequence of a fusion polypeptide of the invention comprises a variant version of an amino acid sequence of ERD10 described herein, where the resulting sequence preserves amino acid composition of the parent sequence.

According to another embodiment of the invention, an EBD sequence of a fusion polypeptide of the invention comprises a variant version of an amino acid sequence of ERD14 described herein, where the resulting sequence preserves amino acid composition of the parent sequence.

In another embodiment, an EBD sequence of a fusion polypeptide of the invention is cleavable, e.g., can be removed and/or separated from the heterologous polypeptide sequence after recombinant expression by, for example, enzymatic or chemical cleavage methods.

In another embodiment, an EBD sequence of a fusion polypeptide of the invention is covalently linked at the N-terminus of the heterologous polypeptide sequence of interest. In another embodiment, an EBD sequence of a fusion polypeptide of the invention is covalently linked at the C-terminus of the heterologous polypeptide sequence of interest. In yet another embodiment, an EBD sequence of a fusion polypeptide of the invention is covalently linked at the N- and C-termini of the heterologous polypeptide sequence of interest.

In another embodiment of the invention, the charge of an EBD sequence of a fusion polypeptide of the invention is modulated by, for example, enzymatic and/or chemical methods, in order to modulate the activity of the EBD sequence. In a particular embodiment, the charge of the EBD sequence is modulated by phosphorylation.

According to another aspect of the invention, an isolated polynucleotide is provided, wherein the polynucleotide encodes a fusion polypeptide as described herein.

According to yet another aspect of the invention, there is provided an expression vector comprising an isolated polynucleotide encoding a fusion polypeptide as described herein. In a related embodiment, an expression vector is provided comprising a polynucleotide encoding an EBD sequence and further comprising a cloning site for insertion of a polynucleotide encoding a heterologous polypeptide of interest.

According to yet another aspect of the invention, there is provided a host cell comprising an expression vector as described herein.

According to yet another aspect of the invention, there is provided a kit comprising an isolated polynucleotide as described herein, an isolated polypeptide as described herein and/or an isolated host cell as described herein.

Yet another aspect of the invention provides a method for producing a recombinant protein comprising the steps of: introducing into a host cell an expression vector comprising a polynucleotide sequence encoding a fusion polypeptide of the invention and expressing the fusion polypeptide in the host cell.

In another embodiment, the method further comprises the step of isolating the fusion polypeptide from the host cell. In another related embodiment, the method further comprises the step of removing the entropic bristle domain sequence from the fusion polypeptide before or after isolating the fusion polypeptide from the host cell.

These and other aspects of the present invention will become apparent upon reference to the following detailed description. All references disclosed herein and in the enclosed Application Data Sheet are hereby incorporated by reference in their entirety as if each was incorporated individually.

### BRIEF DESCRIPTION OF THE DRAWINGS

FIG. 1 shows Western Blots demonstrating the improved solubility of ERD10- and ERD14-fusions with CTLA4.

### BRIEF DESCRIPTION OF THE SEQUENCE IDENTIFIERS

SEQ ID NO: 1 is the amino acid sequence of an A. thaliana ERD10 protein, GenBank accession number NP_{—}564114.

SEQ ID NO: 2 is a polynucleotide sequence encoding the amino acid sequence of SEQ ID NO: 1, GenBank accession number gi I 42562192: base pairs 500-1279.

SEQ ID NO: 3 is the amino acid sequence of an A. thaliana ERD14 protein, GenBank accession number NP_{—}177745.

SEQ ID NO: 4 is a polynucleotide sequence encoding the amino acid sequence of SEQ ID NO: 3, GenBank accession number gi I 42563251: base pairs 221-778.

SEQ ID NO: 5 is the amino acid sequence of a B. napus ERD10 protein, GenBank accession number AAR23753.

SEQ ID NO: 6 is a polynucleotide sequence encoding the amino acid sequence of SEQ ID NO: 5, GenBank accession number gi I 38564508: base pairs 113-928.

### DETAILED DESCRIPTION OF THE INVENTION

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, J., et al. (1989). Molecular Cloning: A Laboratory Manual. 2nd edit, 2. 3 vols, Cold Spring Harbor Laboratory Press, Plainview, N.Y.; Glover, D. M. & Hames, B. D., Eds. (1995). DNA Cloning: A Practical Approach Volume 1: Core Techniques 2 edit. Vol. 1. USA: Oxford Press; Glover, D. M. & Hames, B. D., Eds. (1995). DNA Cloning: A Practical Approach Volume 2: Expression Systems 2 edit. Vol. 2. 3 vols. USA: Oxford University Press; Gait, M. J., Ed. (1984). Oligonucleotide Synthesis: A Practical Approach. USA: Oxford University Press; Hames, B. D. & Higgins, S. J., Eds. (1985). Nucleic Acid Hybridization a Practical Approach. USA: Oxford University Press; Hames, B. D. & Higgins, S. J., Eds. (1984). Transcription and Translation: A Practical Approach. USA: Oxford University Press; Freshney, R. I., Ed. (1992). Animal Cell Culture: A Practical Approach 2 edit. Practical Approach Series. Edited by. USA: Oxford University Press; Perbal, B., Ed. (1988). A Practical Guide to Molecular Cloning. 2 edit. USA: Wiley-Interscience).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

As used herein, the terms “polypeptide” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-expression modifications of the polypeptide, for example, glycosylations, acetylations, phosphorylations and the like, as well as other modifications known in the art, both naturally occurring and non-naturally occurring. Polypeptides of the invention may be prepared using any of a variety of well known recombinant and/or synthetic techniques, illustrative examples of which are further discussed below.

As noted above, the present invention, in a general aspect, relates to the discovery that plant dehydrin sequences are very effective EBD sequences. Dehydrin proteins represent a subfamily of proteins only found in plants, and constitute group 2 of the much larger family of Late Embryogensis Abundant (LEA) proteins. One of the functions of LEA proteins in general and the dehydrins in particular is to mitigate the effects of cellular stress. Several models have been proposed to describe how dehydrin proteins protect cells including acting as ion sinks, membrane stabilization as antioxidants, buffer as hydrate water, and as molecular chaperones (Alsheikh, M. K., Heyen, B. J. & Randall, S. K. (2003). J Biol Chem 278, 40882-9; Koag, M. C et al. (2003). Plant Physio/131, 309-16; Hara, M., et al. (2003). Planta 217, 290-8; Bokor, M. et al. (2005). Biophys J 88, 2030-7; Chakrabortee, S. et al., (2007). Proc Natl Acad Sci USA 104, 18073-8). Evidence consisting of bioinformatic PONDR predictions of disorder and published experimental results indicate that many dehydrin proteins are highly disordered (Mouillon, J. M., Gustafsson, P. & Harryson, P. (2006). Plant Physiol 141, 638-50; Kovacs, D. et al. (2008). Plant Physiol 147, 381-90). The structural disorder of dehydrins such as ERD10 and ERD14 confer several functions that stem from their entropic nature including acting as molecular chaperones in vitro. As demonstrated herein, plant dehydrin sequences provide significant advantages when used in fusion with poorly soluble heterologous polypeptide sequences of interest.

It will be understood that the EBD sequences of the present invention can include dehydrin polynucleotide or polypeptide sequences derived from essentially any plant species known to express dehydrin proteins, illustrative examples of which include Arabidopsis or Brassica. In a more specific embodiment, certain preferred examples include A. thaliana and B. napus. In a more specific embodiment, a plant dehydrin polypeptide sequence used in the present invention comprises at least a fragment of an A. thaliana ERD10 polypeptide sequence set forth in SEQ ID NO: 1, or a variant thereof. In another specific embodiment, the plant dehydrin polypeptide sequence comprises at least a fragment of a B. napus dehydrin ERD10 polypeptide sequence set forth in SEQ ID NO: 5, or variant thereof. In yet another specific embodiment, the plant dehydrin polypeptide sequence comprises at least a fragment of an A. thaliana dehydrin ERD14 polypeptide sequence set forth in SEQ ID NO: 3, or a variant thereof.

By providing an EBD sequence which sweeps out the three-dimensional space surrounding a newly synthesized heterologous polypeptide, the EBD sequences of the invention effectively exclude other polypeptides and thereby minimize aggregation with other newly synthesized heterologous polypeptides during recombinant polypeptide production.

In addition, an EBD sequence of the invention can provide steric stabilization to recombinant polypeptides, a property that is relatively independent of concentration, and can thus minimize problems associated with high-level recombinant production of polypeptides and proteins (e.g., precipitation, toxicity and/or inclusion body formation). Thus, EBD fusion polypeptides described herein exhibit both steric effects (via the entropic bristle's motion) and electrostatic effects (via the bristle's highly charged sequence) to minimize interactions between recombinant polypeptides expressed as fusions according to the present invention. These characteristics allow EBD polypeptide sequences to more effectively solubilize recombinantly expressed polypeptides than, for example, other fusion partners which do not have a steric exclusion component that contributes to their activity.

Therefore, according to one embodiment of the invention, fusion polypeptides comprising an EBD sequence and a heterologous polypeptide are provided which exhibit improved solubility relative to the corresponding heterologous polypeptide in the absence of the EBD sequence. In one embodiment, for example, the fusion polypeptide has at least about 5%, 10%, 15%, 20%, 25% or 30% increased solubility relative to the heterologous polypeptide sequence alone. In another related embodiment, the fusion polypeptide has at least about 50% increased solubility relative to the heterologous polypeptide sequence. In yet another related embodiment, the fusion polypeptide has at least 60% increased solubility relative to the heterologous polypeptide sequence.

The extent of improved solubility provided by an EBD sequence described herein can be determined using any of a number of available approaches (see for example, Kapust, R. B. & Waugh, D. S. (1999) supra; Fox, J. at al. (2003). FEBS Lett 537, 53-7; Dyson, M. R. et al. (2004). BMC Biotechnol 4, 32). For example, cells from single, drug resistant colony of E. coli overproducing the fusion polypeptide are grown to saturation in LB broth (Miller, J. H., Ed. (1972). Experiments in Molecular Genetics. USA: Cold Spring Harbor Laboratory Press) supplemented with 100 mg/mL ampicillin and 30 mg/mL chloramphenicol at 37° C. The saturated cultures are diluted 50-fold in the same medium and grown in shake-flasks to mid-log phase (A_{600˜0.5}-0.7), at which time IPTG is added to a final concentration of 1 mM. After 3 h, the cells are recovered by centrifugation. The cell pellets are resuspended in 0.1 culture volumes of lysis buffer (50 mM Tris-HCl (pH 8.0), 150 mM NaCl, 1 mM EDTA), and disrupted by sonication. A total protein sample is collected from the cell suspension after sonication, and a soluble protein sample is collected from the supernatant after the insoluble debris is pelleted by centrifugation (20,000×g). These samples are subjected to SDS-PAGE and proteins are visualized by staining with Coomassie Brilliant Blue. At least three independent experiments are typically performed to obtain numerical estimates of the solubility of each fusion protein in E. coli. Coomassie-stained gels will be scanned with a gel-scanning densitometer and the pixel densities of the bands corresponding to the fusion proteins are obtained directly by volumetric integration. In each lane, the collective density of all E. coli proteins that are larger than the largest fusion protein are also determined by volumetric integration and used to normalize the values in each lane relative to the others. The percent solubility of each fusion protein is calculated by dividing the amount of soluble fusion protein by the total amount of fusion protein in the cells, after first subtracting the normalized background values obtained from negative control lanes (cells containing no expression vector). Descriptive statistical data (e.g., the mean and standard deviation) is then generated using standard methods.

The presence of an EBD sequence in fusion polypeptides of the present invention can also serve to reduce the extent of aggregation of a heterologous polypeptide sequence. In one embodiment, for example, the fusion polypeptide exhibits at least about 5%, 10%, 15%, or 20% reduced aggregation relative to the heterologous polypeptide. In another embodiment, the fusion polypeptide has at least 25% reduced aggregation relative to the heterologous polypeptide.

The extent of reduced aggregation provided by the fusion polypeptides of the present invention can be determined using any of a number of available techniques (see for example (Philo, J. S. (2009). Curr Pharm Biotechnol 10, 359-72; Wang, W. & Roberts, C. J., Eds. (2010). Aggregation of Therapeutic Proteins. USA: Wiley; Munoz, V., Ed. (2008). Protein Folding, Misfolding and Aggregation: Classical Themes and Novel Approaches: Royal Society of Chemistry; Uversky, V. N. & Fink, A., Eds. (2006). Protein Misfolding, Aggregation and Conformational Diseases: Part A: Protein Aggregation and Conformational Diseases USA: Springer). Briefly, protein aggregates vary widely in both size and structure, but can be broadly divided into 2 classes, particulate and “soluble” aggregates. Particulate forming aggregates are often detected by methods such as particle counting and microscopy. For soluble aggregates size-exclusion chromatography is the most widely used technique for quantitation of size. However, methods including dynamic light scattering, analytical ultracentrifugation, and field flow fractionation are valuable alternative methods to consider. Further analysis of the secondary and tertiary structure of the soluble aggregate can be measured using methods such as FT-IR and Raman spectroscopy.

The presence of an EBD sequence in the fusion polypeptides of the present invention can also serve to improve the folding characteristics of the fusion polypeptides relative to the corresponding heterologous polypeptide, e.g., by minimizing interference caused by interaction with other proteins.

Assays for evaluating the folding characteristics of a fusion polypeptide of the invention can be carried out using conventional techniques, such as circular dichroism spectroscopy in far ultra-violet region, circular dichroism in near ultra-violet region, nuclear magnetic resonance spectroscopy, infra-red spectroscopy, Raman spectroscopy, intrinsic fluorescence spectroscopy, extrinsic fluorescence spectroscopy, fluorescence resonance energy transfer, fluorescence anisotropy and polarization, steady-state fluorescence, time-domain fluorescence, numerous hydrodynamic techniques including gel-filtration, viscometry, small-angle X-ray scattering, small angle neutron scattering, dynamic light scattering, static light scattering, scanning microcalorimetry, and limited proteolysis.

In another embodiment of the invention, an EBD comprises an amino acid sequence that maintains a substantially random coil conformation. Whether a given amino acid sequence maintains a substantially random coil conformation can be determined by circular dichroism spectroscopy in far ultra-violet region, nuclear magnetic resonance spectroscopy, infra-red spectroscopy, Raman spectroscopy, fluorescence spectroscopy, numerous hydrodynamic techniques including gel-filtration, viscometry, small-angle X-ray scattering, small angle neutron scattering, dynamic light scattering, static light scattering, scanning microcalorimetry, and limited proteolysis.

In another embodiment of the invention, an EBD sequence comprises an amino acid sequence that is substantially mutually repulsive. As is known in the art, this property of being mutually repulsive can be determined by simple calculations of charge distribution within the polypeptide sequence.

In yet another embodiment of the invention, an EBD sequence comprises an amino acid sequence that remains in substantially constant motion, particularly in an aqueous environment. The property of being in substantially constant motion can be determined by nuclear magnetic resonance spectroscopy, small-angle X-ray scattering, small angle neutron scattering, dynamic light scattering, intrinsic fluorescence spectroscopy, extrinsic fluorescence spectroscopy, fluorescence resonance energy transfer, fluorescence anisotropy and polarization, steady-state fluorescence, time-domain fluorescence.

According to a preferred embodiment of the present invention, an EBD sequence used in the context of the present invention is derived from a plant dehyrdin protein. In a more specific embodiment, the plant dehyrdin protein is an ERD10 and/or ERD14 plant dehydrin protein, or a related protein upregulated in response to cellular stress (including in A. thaliana and B. napus). As will be understood by those skilled in the art, the propensity of a polypeptide chain to maintain a substantially random coil and flexible conformation is encoded in its amino acid composition rather than in its amino acid sequence (Uversky, V. N. et al. (2000). Proteins 41, 415-27). This means that “natively unfolded” polypeptides sharing similar amino acid compositions will be similarly unfolded. The function of EBDs to increase protein solubility is based at least in part on their random coil and flexible conformation. Therefore, in one embodiment of the invention, an EBD sequence of the invention comprises a scrambled variant sequence of a plant ERD10 protein. In another embodiment of the invention, an EBD sequence of the invention comprises a scrambled variant sequence of a plant ERD14 protein.

In another embodiment of the invention, an EBD sequence of the invention comprises a scrambled variant sequence corresponding to any combination of fragments derived from sequence of a plant ERD10 protein. In yet another embodiment of the invention, an EBD sequence of the invention comprises a scrambled variant sequence corresponding to any combination of fragments derived from sequence of a plant ERD14 protein.

In another embodiment of the invention, an EBD sequence of the invention comprises a scrambled variant sequence corresponding to multiple repeats of any combination of fragments derived from sequence of a plant ERD10 protein. In yet another embodiment of the invention, an EBD sequence of the invention comprises a scrambled variant sequence corresponding to multiple repeats of any combination of fragments derived from sequence of a plant ERD14.

In another embodiment of the invention, an EBD sequence of the invention comprises a scrambled variant sequence corresponding to any pairwise or multiple combinations of fragments derived from sequence of a plant ERD10 protein and a plant ERD14 protein.

In yet another embodiment of the invention, an EBD sequence of the invention comprises a scrambled variant sequence corresponding to multiple repeats of any pairwise or multiple combinations of fragments derived from sequence of a plant ERD10 protein and a plant ERD14 protein.

In another embodiment, the fusion polypeptides of the invention further comprise independent cleavable linkers, which allow an EBD sequence, for example at either the N or C terminus, to be easily cleaved from a heterologous polypeptide sequence of interest. Such cleavable linkers are known and available in the art. This embodiment thus provides improved isolation and purification of a heterologous polypeptide sequence and facilitates downstream high-throughput processes.

In certain preferred embodiments, an EBD sequence used in the present invention is a full length plant dehydrin polypeptide sequence, such as an ERD10 polypeptide sequence or an ERD14 polypeptide sequence, or a fragment or variant thereof. In more specific embodiments, the ERD10 and/or ERD14 polypeptide sequence is derived from a plant species selected from Arabidopsis or Brassica.

In a more specific embodiment, a plant dehydrin polypeptide sequence used in the present invention comprises at least a fragment of an A. thaliana ERD10 polypeptide sequence set forth in SEQ ID NO: 1, or a variant thereof. In another specific embodiment, the plant dehydrin polypeptide sequence comprises at least a fragment of a B. napus dehydrin ERD10 polypeptide sequence set forth in SEQ ID NO: 5, or variant thereof. In yet another specific embodiment, the plant dehydrin polypeptide sequence comprises at least a fragment of an A. thaliana dehydrin ERD14 polypeptide sequence set forth in SEQ ID NO: 3, or a variant thereof.

As noted, the present invention provides polypeptide fragments of an EBD polypeptide sequence described herein, wherein the fragment comprises at least about 5, 10, 15, 20, 25, 50, 100, 150, 200, 250, 300, 350, 400, 450, 500, 600, 700, 800, 900, 1000, etc., contiguous amino acids, or more, including all intermediate lengths, of an EBD polypeptide sequence set forth herein, such as the plant dehydrin polypeptide sequences described herein, or those encoded by a polynucleotide sequence set forth herein. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. Preferably, an EBD polypeptide fragment retains one or more desired activities, e.g., improved solubility, improved folding, reduced aggregation and/or improved yield, when in fusion with a heterologous sequence of interest.

As also noted, the present invention provides variants of an EBD polypeptide sequence, such as the plant dehydrin polypeptide sequences described herein, or those encoded by a polynucleotide sequence set forth herein. EBD polypeptide variants will typically exhibit at least about 70%, 75%, 80%, 85%, 90%, 91%, 92%, 93%, 94%, 95%, 96%, 97%, 98%, or 99% or more identity, including all intermediate percent identities, (e.g., determined as described below), along its length, to an EBD polypeptide sequence described herein (e.g., a plant dehydrin sequence set forth in SEQ ID NOs: 1, 3, 5, etc.). Preferably the EBD variant provides similar or improved activity relative to the activity of the EBD sequence from which the variant was derived (wherein the activity includes one or more of improved solubility, improved folding, reduced aggregation and/or improved yield, when in fusion with a heterologous polypeptide sequence of interest.

An EBD polypeptide variant thus refers to a polypeptide that differs from an EBD polypeptide sequence disclosed herein in one or more substitutions, deletions, additions and/or insertions. Such variants may be naturally occurring or may be synthetically generated, for example, by modifying one or more of the EBD polypeptide sequences of the invention and evaluating their activity as described herein and/or using any of a number of techniques well known in the art.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the EBD polynucleotides and polypeptides of the present invention and still obtain a functional molecule that encodes a variant or derivative polypeptide with desirable activity. When it is desired to alter the amino acid sequence of an EBD polypeptide to create an equivalent or an improved EBD variant or EBD fragment, one skilled in the art can readily change one or more of the codons of the encoding DNA sequence, for example according to Table 1.

For example, certain amino acids may be substituted for other amino acids in a protein structure without appreciable loss of desired activity. It is thus contemplated that various changes may be made in the EBD polypeptide sequences of the invention, or corresponding DNA sequences which encode said EBD polypeptide sequences, without appreciable loss of their desired activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may also be considered. The importance of the hydropathic amino acid index in conferring interactive biologic function on a protein is generally understood in the art (Kyte, J. & Doolittle, R. F. (1982). J. Mol. Biol. 157, 105-132) incorporated herein by reference). It is accepted that the relative hydropathic character of the amino acid contributes to the secondary structure of the resultant protein, which in turn has potential bearing on the interaction of the protein with other molecules, for example, enzymes, substrates, receptors, DNA, antibodies, antigens, and the like. Each amino acid has been assigned a hydropathic index on the basis of its hydrophobicity and charge characteristics (ibid). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Therefore, according to certain embodiments, amino acids within an EBD sequence of the invention may be substituted by other amino acids having a similar hydropathic index or score. Preferably, any such changes result in an EBD sequence with a similar level of activity as the unmodified EBD sequence. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Thus, an amino acid can be substituted for another having a similar hydrophilicity value and in many cases still retain a desired level of activity. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like.

In addition, any polynucleotide of the invention, such as a polynucleotide encoding an EBD polypeptide sequence, or a vector comprising a polynucleotide encoding an EBD polypeptide sequence, may be further modified to increase stability in viva Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Amino acid substitutions within an EBD sequence of the invention may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

In an illustrative embodiment, a variant EBD polypeptide differs from the corresponding unmodified EBD sequence by substitution, deletion or addition of five percent of the original amino acids or fewer. Variants may also (or alternatively) be modified by, for example, the deletion or addition of amino acids that have minimal influence on the desired activity.

A polypeptide of the invention may further comprise a signal (or leader) sequence at the N-terminal end of the polypeptide, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support.

As noted above, the present invention provides EBD polypeptide variant sequences which share some degree of sequence identity with an EBD polypeptide specifically described herein. When comparing polypeptide sequences to evaluate their extent of shared sequence identity, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O., Schwartz, R. M. & Orcutt, B. C. (1978). A model of evolutionary change in proteins. Atlas of Protein Sequence and Structure 5 Supp 3, National Biomedical Research Foundation, Silver Spring; Hein, J. (1990). Methods Enzymol 183, 626-45; Higgins, D. G. & Sharp, P. M. (1989). Comput Appl Biosci 5, 151-3; Myers, E. W. & Miller, W. (1988). Comput Appl Biosci 4, 11-7; Robinson, D. F. (1971). J. Comb. Theor. 11, 105-119; Saitou, N. & Nei, M. (1987). Mol. Biol. Evol. 4, 406-25; Sneath, P. H. A. & Sokal, R. R. (1973). Numerical taxonomy: the principles and practice of numerical classification, Freeman San Francisco; Wilbur, W. J. & Lipman, D. J. (1983). Proc Natl Acad Sci USA 80, 726-30). Alternatively, optimal alignment of sequences for comparison may be conducted by the local identity algorithm of Smith and Waterman, by the identity alignment algorithm of Needleman and Wunsch, by the search for similarity methods of Pearson and Lipman, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection (Smith, T. F. & Waterman, M. S. (1981). Advances in Applied Mathematics 2, 482-489; Needleman, S. B. & Wunsch, C. D. (1970). J. Mol. Biol. 48, 443-453; Pearson, W. R. & Lipman, D. J. (1988). Proc Nat/Acad Sci USA 85, 2444-8).

One preferred example of algorithms that are suitable for determining percent sequence identity and sequence similarity are the BLAST and BLAST 2.0 algorithms, which are described by Altschul (Altschul, S. F et al. (1990). J. Mol. Biol. 215, 403-410; Altschul, S. F. et al., (1997). Nucleic Acids Res. 25, 3389-3402). BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

According to another aspect of the invention, there is provided an isolated polynucleotide sequence encoding a fusion polypeptide, the fusion polypeptide comprising at least one EBD sequence as described herein, such as a plant dehydrin polypeptide sequence or fragment or variant thereof, and at least one heterologous polypeptide sequence of interest. In a related aspect, the invention provides expression vectors comprising a polynucleotide encoding an EBD fusion polypeptide of the invention. In another related aspect, an expression vector of the invention comprises a polynucleotide encoding one or more EBD sequence and further comprises a multiple cloning site for the insertion of a polynucleotide encoding a heterologous polypeptide sequence of interest.

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references).

The terms “DNA” and “polynucleotide” are used essentially interchangeably herein to refer to a DNA molecule that has been isolated free of total genomic DNA of a particular species. “Isolated”, as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will also be recognized, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

In addition to the EBD polynucleotide sequences set forth herein, the present invention also provides EBD polynucleotide variants having substantial identity to an EBD polynucleotide sequence disclosed herein, for example those comprising at least 50% sequence identity, preferably at least, 55%, 60%, 65%, 70%, 75%, 80%, 85%, 90%, 95%, 96%, 97%, 98%, or 99% or higher, including all intermediate percent identities, sequence identity compared to an EBD polynucleotide sequence of this invention using the methods described herein, (e.g., BLAST analysis using standard parameters, as described below). One skilled in this art will recognize that these values can be appropriately adjusted to determine corresponding identity of polypeptides encoded by two polynucleotide sequences by taking into account codon degeneracy, amino acid similarity, reading frame positioning and the like.

Typically, EBD polynucleotide variants will contain one or more substitutions, additions, deletions and/or insertions, preferably such that the activity (e.g., improved folding, reduced aggregation and/or improved yield, when in fusion with a heterologous sequence of interest) of the polypeptide encoded by the variant polynucleotide is not substantially diminished relative to the corresponding unmodified polynucleotide sequence.

In additional embodiments, the present invention provides polynucleotide fragments comprising or consisting of various lengths of contiguous stretches of sequence identical to or complementary to one or more of the EBD polynucleotide sequences disclosed herein. For example, polynucleotides are provided by this invention that comprise or consist of at least about 10, 15, 20, 30, 40, 50, 75, 100, 150, 200, 300, 400, 500, 600, 700, 800, 900, 1000, 2000, etc., or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-300; 300-400, 500-600, 600-700, 700-800, 800-900, 900-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence. Preferably, an EBD polynucleotide fragment of the invention encodes a fusion polypeptide that retains one or more desired activities, e.g., improved folding, reduced aggregation and/or improved yield, when in fusion with a heterologous sequence of interest.

The EBD polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

It will be appreciated by those of ordinary skill in the art that, as a result of the degeneracy of the genetic code, there are many nucleotide sequences that will encode a polypeptide as described herein. Some of these polynucleotides bear minimal homology to the native polynucleotide sequence. Nonetheless, polynucleotides that vary due to differences in codon usage are specifically contemplated by the present invention. Further, different alleles of an EBD polynucleotide sequence provided herein are within the scope of the present invention. Alleles are endogenous sequences that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

In another embodiment of the invention, a mutagenesis approach, such as site-specific mutagenesis, may be employed for the preparation of variants and/or derivatives of the EBD polynucleotides and polypeptides described herein. By this approach, for example, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the present invention contemplates the mutagenesis of the disclosed polynucleotide sequences to alter one or more activities/properties of the encoded polypeptide. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length may be employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of (Maloy, S. R. et al. (1994). Microbial Genetics 2 edit. Jones and Bartlett Publishers, Sudbury; Prokop, A. et al., Eds. (1991). Recombinant DNA Technology and Applications Texas: McGraw-Hill; Sambrook, J. et al. (1989). Supra), each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, J. D. et al. Eds. (2007). Molecular Biology of the Gene. 6 edit. USA: Benjamin Cummings). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention wherein one or more desired activities is improved or modified.

In other embodiments of the present invention, the polynucleotide sequences provided herein can be advantageously used as probes or primers for nucleic acid hybridization. As such, it is contemplated that nucleic acid segments that comprise or consist of a sequence region of at least about a 15 nucleotide long contiguous sequence that has the same sequence as, or is complementary to, a 15 nucleotide long contiguous sequence disclosed herein may be used. Longer contiguous identical or complementary sequences, e.g., those of about 20, 30, 40, 50, 100, 200, 500, 1000 (including all intermediate lengths) and even up to full length sequences will also be of use in certain embodiments.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE”, and “one-sided PCR” are also well-known to those of skill in the art (Frohman, M. A. (1990). PCR protocols: A guide to methods and applications (Innis, M. A., et al., Eds.), Academic Press, San Diego; Ohara, O. et al. (1989). Proc Natl Acad Sci USA 86, 5673-5677).

As noted, the EBD fusion polynucleotides, polypeptides and vectors of the present invention are advantageous in the context of recombinant polypeptide production, particularly where it is desired to achieve, for example, improved solubility, improved yield, improved folding and/or reduced aggregation of a heterologous polypeptide to which an EBD polypeptide sequence has been operably fused. Therefore, another aspect of the invention provides methods for producing a recombinant protein, for example by introducing into a host cell an expression vector comprising a polynucleotide sequence encoding a fusion polypeptide as described herein, e.g., a fusion polypeptide comprising at least one EBD sequence and at least one heterologous polypeptide sequence of interest; and expressing the fusion polypeptide in the host cell. In a related embodiment, the method further comprises the step of isolating the fusion polypeptide from the host cell. In another embodiment, the method further comprises the step of removing an entropic bristle domain sequence from the fusion polypeptide before or after isolating the fusion polypeptide from the host cell.

For recombinant production of a fusion polypeptide of the invention, DNA sequences encoding the polypeptide components of a fusion polypeptide (e.g., one or more EBD sequences and a heterologous polypeptide sequence of interest) may be assembled using conventional methodologies. In one example, the components may be assembled separately and ligated into an appropriate expression vector. For example, the 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the activities of both component polypeptides.

A peptide linker sequence may be employed to separate an EBD polypeptide sequence from a heterologous polypeptide sequence by some defined distance, for example a distance sufficient to ensure that the advantages of the invention are achieved, e.g, advantages such as improved folding, reduced aggregation and/or improved yield. Such a peptide linker sequence may be incorporated into the fusion polypeptide using standard techniques well known in the art. Suitable peptide linker sequences may be chosen based, for example, on the factors such as: (1) their ability to adopt a flexible extended conformation; and (2) their inability to adopt a secondary structure that could interfere with the activity of the EBD sequence. Illustrative peptide linker sequences, for example, may contain Gly, Asn and Ser residues. Other near neutral amino acids, such as Thr and Ala may also be used in the linker sequence. Amino acid sequences which may be usefully employed as linkers include those disclosed in Maratea et al. (Maratea, D. et al., (1985). Gene 40, 39-46; U.S. Pat. No. 4,935,233 and U.S. Pat. No. 4,751,180). The linker sequence may generally be from 1 to about 50 amino acids in length, for example.

The ligated DNA sequences of a fusion polynucleotide are operably linked to suitable transcriptional and/or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The EBD and heterologous polynucleotide sequences may comprise a sequence as described herein, or may comprise a sequence that has been modified to facilitate recombinant polypeptide production. As will be understood by those of skill in the art, it may be advantageous in some instances to produce polypeptide-encoding polynucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In a particular embodiment, a fusion polynucleotide is engineered to further comprise a cleavage site located between the EBD polypeptide-encoding sequence and the heterologous polypeptide sequence, so that the hetereolous polypeptide may be cleaved and purified away from an EBD polypeptide sequence at any desired stage following expression of the fusion polypeptide. Illustratively, a fusion polynucleotide of the invention may be designed to include heparin, thrombin, or factor Xa protease cleavage sites.

In order to express a desired polypeptide, the nucleotide sequences encoding the polypeptide, or functional equivalents, may be inserted into appropriate expression vector, i.e., a vector which contains the necessary elements for the transcription and translation of an inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook et al. and Ausubel et al. (Sambrook, J. et al. (1989) supra; and Ausubel, F. M. et al. Eds. (1988). Current Protocols in Molecular Biology Vol. 3. New York: John Wiley and Sons).

A variety of expression vector/host systems may be utilized to contain and express polynucleotide sequences of the present invention. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. & Schuster, S. M. (1989). J Biol Chem 264, 19475-7); and the like. Proteins made in such systems may be designed to include heparin, thrombin, or factor Xa protease cleavage sites so that the cloned polypeptide of interest can be released from the EBD moiety at will.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. et al. (1987). Embo J 6, 307-11). Alternatively, plant promoters such as the small subunit of RUBISCO or heat shock promoters may be used (Coruzzi, G. et al. (1984). Embo J 3, 1671-9; Broglie, R. et al. (1984). Science 224, 838-43; Winter, J. & Sinibaldi, R. (1991). Results Probl Cell Differ 17, 85-105). These constructs can be introduced into plant cells by direct DNA transformation or pathogen-mediated transfection. Such techniques are described in a number of generally available reviews (see, for example, Murry, L. E. (1992). Agrobacterium-Mediated plant transformation. McGraw Hill Yearbook of Science and Technology, McGraw Hill, New York. NY).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994). Proc Natl Acad Sci USA 91, 3224-7).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. & Shenk, T. (1984). Proc Natl Acad Sci USA 81, 3655-9). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, K. D. et al. (1994). Results Probl Cell Differ 20, 125-62).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase and adenine phosphoribosyltransferase genes which can be employed in tk.sup.- or aprt.sup.- cells, respectively (Wigler, M. et al. (1977). Cell 11, 223-32; Lowy, I. et al. (1980). Cell 22, 817-23). Also, antimetabolite, antibiotic or herbicide resistance can be used as the basis for selection; for example, dhfr which confers resistance to methotrexate; npt, which confers resistance to the aminoglycosides, neomycin and G-418; and als or pat, which confer resistance to chlorsulfuron and phosphinotricin acetyltransferase, respectively (Wigler, M. et al. (1980). Proc Natl Acad Sci USA 77, 3567-70; Colbere-Garapin, F. et al (1981). J Mol Biol 150, 1-14; Murry, L. E. (1992) supra). Additional selectable genes have been described, for example, trpB, which allows cells to utilize indole in place of tryptophan, or hisD, which allows cells to utilize histinol in place of histidine (Hartman, S. C. & Mulligan, R. C. (1988). Proc Natl Acad Sci USA 85, 8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995). Methods Mol Biol 55, 121-31).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton and Maddox (Hampton, R., Ball, E. & DeBoar, S., Eds. (1990). Serological Methods for Detection and Identification of Viral and Bacterial Plant Pathogens: Laboratory Manual. St Paul: American Phytopathological Society; Maddox, D. E. et al. (1983). J Exp Med 158, 1211-26). A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the polypeptide from cell culture. The polypeptide produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to polynucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor Xa or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath et al., while the enterokinase cleavage site provides a means for purifying the desired polypeptide from the fusion protein (Porath, J. (1992). Protein Expr Purif 3, 263-81). Further discussion of vectors which comprise fusion proteins can be found in Kroll (Kroll, D. J. et al. (1993) DNA Cell Biol 12, 441-53).

In addition to recombinant production methods, polypeptides of the invention, and fragments thereof, may be produced by direct peptide synthesis using solid-phase techniques (Merrifield, R. B. (1963). Solid Phase Peptide Synthesis. I. The Synthesis of a Tetrapeptide. Journal of the American Chemical Society 85, 2149-2154). Polypeptide synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

According to another aspect, the present invention further provides binding agents, such as antibodies and antigen-binding fragments thereof, that specifically bind to an EBD sequence according to the present invention, or to a portion, variant or derivative thereof. Such binding agents may be used, for example, to detect the presence of a polypeptide comprising an EBD sequence, to facilitate purification of a polypeptide comprising an EBD sequence, and the like. An antibody, or antigen-binding fragment thereof, is said to “specifically bind” to a polypeptide if it reacts at a detectable level (within, for example, an ELISA assay) with the polypeptide, and does not react detectably with unrelated polypeptides under similar conditions.

Antibodies and other binding agents can be prepared using conventional methodologies. For example, monoclonal antibodies specific for a polypeptide of interest may be prepared using the technique of Kohler and Milstein, and improvements thereto (Kohler, G. & Milstein, C. (1976). Eur J Immunol 6, 511-9). Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described, including chimeric antibodies having rodent V regions and their associated CDRs fused to human constant domains, rodent CDRs grafted into a human supporting FR prior to fusion with an appropriate human antibody constant domain, and rodent CDRs supported by recombinantly veneered rodent FRs (Winter, G. & Milstein, C. (1991). Nature 349, 293-9; LoBuglio, A. F. et al. (1989). Proc Natl Acad Sci USA 86, 4220-4; Shaw, D. R. et al., (1987). J Immuno/138, 4534-8; Brown, B. A., et al. (1987). Cancer Res 47, 3577-83; Riechmann, L. et al., (1988). Nature 332, 323-7; Verhoeyen, M. et al. (1988). Science 239, 1534-6; Jones, P. T. et al. (1986). Nature 321, 522-5; and European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

Yet another aspect of the invention provides kits comprising one or more compositions described herein, e.g., an isolated EBD polynucleotide, polypeptide, antibody, vector, host cell, etc. In a particular embodiment, the invention provides a kit containing an expression vector comprising a polynucleotide sequence encoding an EBD polypeptide sequence and a multiple cloning site for easily introducing into the vector a polynucleotide sequence encoding a heterologous polypeptide sequence of interest. In another embodiment, the expression vector further comprises an engineered cleavage site to facilitate separation of an EBD polypeptide sequence from the hetereologous polypeptide sequence of interest following recombinant production.

The following Examples are offered by way of illustration and not by way of limitation.

### EXAMPLES

### Example 1

### Fusion Polypeptides Comprising Plant Dehydrin Protein Sequences

Late Embryogensis Abundant (LEA) proteins accumulate in organisms during development and in response to cellular stresses such as cold stress and dehydration. The LEA proteins constitute as large and diverse family of divergent proteins that are separated into three groups by the presence of certain sequence motifs. Group 2 proteins are only found in plants and are also known as dehydrins. A number of protective functions have been proposed to describe how dehydrins work including acting as ion sinks, through membrane stabilization, as antioxidants, as a buffer of hydrate water, and as molecular chaperones (Alsheikh, M. K., Heyen, B. J. & Randall, S. K. (2003). J Biol Chem 278, 40882-9; Koag, M. C et al. (2003). Plant Physiol 131, 309-16; Hara, M., et al. (2003). Planta 217, 290-8; Bokor, M. et al. (2005). Biophys J 88, 2030-7; Chakrabortee, S. et al. (2007). Proc Natl Acad Sci USA 104, 18073-8). Furthermore, most dehydrin proteins have been shown to be intrinsically disordered (Mouillon, J. M., Gustafsson, P. & Harryson, P. (2006). Plant Physiol 141, 638-50; Kovacs, D. et al. (2008). Plant Physiol 147, 381-90).

At least six dehydrin proteins are encoded by the A. thaliana genome. Bioinformatic analyses indicated that five out of the six dehydrins are predicted to be highly disordered, and likely to maintain a dynamic random coil confirmation in solution (Table 2.). In addition, three of the Arabidopsis dehydrin proteins have an overall net negative charge (ERD10, ERD14, and COR47) (Table 2), which can be an important parameter contributing to protein solubility.

TABLE 2
Plant Dehydrin EBD Properties.

| | | | | | | Ave. |
|---|---|---|---|---|---|---|
| | Accession | MW | | | Percent | Pred |
| GeneID | Number | (kD) | Charge | Length | Disordered | Score |
| ERD10 | NP_561114 | 29.4 | −15 | 259 | 64% | 0.5863 |
| ERD14 | NP_177745 | 20.8 | −9 | 185 | 64% | 06123 |
| COR47 | NP_173468 | 29.9 | −35 | 265 | 64% | 0.5862 |
| RAB18 | CAA48178 | 18.5 | 0 | 186 | 80% | 0.7081 |
| XERO1 | NP_190667 | 13.4 | +3 | 128 | 60% | 0.5939 |
| LTI30 | NP_190666 | 20.9 | +6 | 193 | 21% | 0.2646 |

We evaluated certain plant dehydrin protein sequences as possible EBD sequences for use in fusion polypeptides for improving recombinant production of proteins having low solubility, as set out below.

Cloning of EBD Sequence

The coding region of a full length A. thaliana ERD10 cDNA was cloned into a pET45b+ vector in between the coding sequences for a 6×His-tag and an enterokinase cleavage site. The endogenous pET45b+ multicloning site was maintained downstream of the enterokinase cleavage site coding sequence. A heterologous protein expressed from this modified expression plasmid therefore has an N-terminal fusion tag consisting of 6×His-ERD10 that can be removed following cleavage with enterokinase. This construct is referred to as pET-ERD10. Similar constructs were also prepared with other A. thaliana dehydrin proteins, including ERD14, Rab18 and LTI30 (a.k.a., XERO2).

Preparation of Heterologous Sequence

The coding region of a heterologous sequence of interest may be examined for rare E. coli codons and restrictions sites for a suitable cloning strategy. Prior to cloning, incompatible codons and restriction sites may be altered by site directed mutagenesis. The heterologous protein coding region, not including the stop codon, is PCR-amplified using primers containing the relevant restriction sites for the 5′ and the 3′ ends of the test protein open reading frame respectively.

Assembly of EBD Expression Vector

The PCR-amplified open reading frame of the heterologous polypeptide sequence of interest was cloned into the pET-ERD10 vector backbone following digestion with appropriate restriction enzymes. In addition to cloning the heterologous sequence into an EBD expression vector, the test proteins were also cloned into an MBP expression vector (e.g., pET-MBP, which contains a maltose-binding protein coding region in place of ERD10) as well as a control vector. In these experiments, the pET-MBP vector served as a control for solubility improvement and the parent pET45b+ vector served as a negative control, as the 6×His-tag fusion does not confer improved solubility to the protein to which it is attached.

Protein expression and solubility analysis were carried out as follows. Briefly, the construct was transformed into E. coli Acella cells (EdgeBio, Gaithersburg Md.). The transformed cells were grown at 37° C. with shaking in LB broth supplemented with the appropriate antibiotics, diluted 30 fold into LBSB medium, and grown for 2 hrs prior to induction. Recombinant protein production was induced with IPTG at a final concentration of 0.2 mM for 4-6 hours at 25° C., and harvested by centrifugation. The pellets were resuspended in 0.3 ml of B-Per lysis buffer (Pierce, Rockford Ill.) containing DNAse I and vortexed to disrupt cells. A sample of this crude lysate was reserved and used for total protein analyses. After the crude lysate was cleared by centrifugation, a sample of the cleared lysate was used for soluble protein analyses. The insoluble pellet was resuspended in 0.3 ml of lysis buffer and a sample was used for insoluble protein analyses. These samples were run on SDS-PAGE gels using standard procedures and transferred to PVDF membranes. The protein gel blots were probed with anti-His probe antibodies, washed, probed with secondary antibodies that were conjugated with alkaline phosphatase, washed, and visualized following incubation in alkaline phosphatase chromogen. The recombinant protein was apparent as a purple stained band.

The dried protein gel blots were scanned using an Epson Perfection 4490 scanner (Epson, Long Beach, Calif.) and the density of the protein bands was quantified using NIH ImageJ analysis software (NIH, http://rsb.info.nih.gov/ij/). The densities of the bands corresponding to the fusion protein in the soluble and insoluble lanes were normalized by subtracting background signal. Percent solubility was calculated by summing the soluble and insoluble band densities and dividing the normalized density of the fusion protein band in the cleared lysate (soluble protein) lane by the total calculated density.

Results

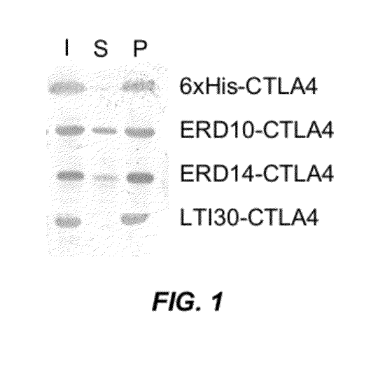

The effectiveness of ERD10, ERD14, and LTI30 as solubility enhancing EBD fusion tags was evaluated following the protocols described for a test set of five proteins. In general ERD10 and ERD14 improved the solubility of a heterologously expressed protein partner when compared to the control 6×His fusion protein. The results for Western blot results for CTLA4 are shown in FIG. 1 and demonstrate that both ERD10 and ERD14 improved solubility of this protein. In contrast, LTI30 failed to improve solubility and in some instances diminished solubility when compared to the 6×His fusion control (FIG. 1).

A larger set of proteins was cloned into the pET-ERD10, pET-MBP, and pET45b+ expression vectors to allow for comparison between a soluble structured fusion protein (MBP) and the intrinsically disordered ERD10 fusion. The 6×His-, MBP-, and ERD10 fusion proteins were expressed in triplicate and the percentage of fusion protein in the soluble fraction was determined. The mean and standard deviation was determined and presented in Table 3. Among the test set of 10 insoluble heterologous proteins, ERD10 was found to be superior to MBP, the current market leader in solubility-enhancing translational fusion products.

TABLE 3

| Target | 6xHis | MBP | ERD10 |
|---|---|---|---|
| IL-13 | 4% ± 8% | 39% ± 3% | 64% ± 23% |
| IL-21 | 0% ± 0% | 7% ± 6% | 31% ± 6% |
| CaMKII | 0% ± 0% | 0% ± 0% | 34% ± 10% |
| c-Src | 5% ± 4% | 6% ± 5% | 12% ± 1% |
| CTLA4 | 8% ± 7% | 18% ± 8% | 32% ± 4% |
| EFNA1 | 0% ± 0% | 0% ± 0% | 25% ± 7% |
| MAD | 0% ± 1% | 21% ± 2% | 50% ± 3% |
| MSTNmature | 4% ± 3% | 18% ± 7% | 33% ± 21% |
| TEV | 11% ± 9% | 61% ± 2% | 75% ± 5% |
| TIMP2 | 0% ± 1% | 3% ± 4% | 58% ± 30% |

These results demonstrate that plant dehydrin sequences can not only be effectively used as EBD sequences for improving the solubility of heterologous proteins, they can improve the solubility of heterologous proteins more effectively than other known solubility-enhancing sequences, such as MBP.

1. An isolated fusion polypeptide comprising a plant dehydrin polypeptide sequence covalently linked to a heterologous polypeptide sequence, wherein the fusion polypeptide has increased solubility, reduced aggregation and/or improved folding relative to the heterologous polypeptide sequence.

2. The fusion polypeptide according to claim 1, wherein the plant dehydrin polypeptide sequence comprises a plant dehydrin ERD10 polypeptide sequence.

3. The fusion polypeptide according to claim 1, wherein the plant dehydrin polypeptide sequence comprises at least a fragment of an A. thaliana ERD10 polypeptide sequence set forth in SEQ ID NO: 1, or a sequence having at least 90% identity thereto.

4. The fusion polypeptide according to claim 1, wherein the plant dehydrin polypeptide sequence comprises a B. napus dehydrin ERD10 protein.

5. The fusion polypeptide according to claim 1, wherein the plant dehydrin polypeptide sequence comprises at least a fragment of a B. napus dehydrin ERD10 polypeptide sequence set forth in SEQ ID NO: 5, or a sequence having at least 90% identity thereto.

6. The fusion polypeptide according to claim 1, wherein the plant dehydrin polypeptide sequence comprises a plant dehydrin ERD14 polypeptide sequence.

7. The fusion polypeptide according to claim 1, wherein the plant dehydrin polypeptide sequence comprises at least a fragment of an A. thaliana dehydrin ERD14 polypeptide sequence set forth in SEQ ID NO: 3, or a sequence having at least 90% identity thereto.

8. The fusion polypeptide according to claim 1, wherein the polypeptide further comprises a cleavable linker.

9. An isolated polynucleotide encoding a fusion polypeptide according to claim 1.

10. An expression vector comprising an isolated polynucleotide according to claims 9.

11. A host cell comprising an expression vector according to claim 10.

12. A kit comprising an isolated polynucleotide according to claim 11.

13. A kit comprising an expression vector according to claim 11.

14. A kit comprising a host cell according to claim 11.

15. A method for producing a recombinant protein comprising the steps of: (a) introducing into a host cell a polynucleotide according to claim 9 or an expression vector according to claim 10; and (b) expressing in the host cell a fusion polypeptide comprising a plant dehydrin polypeptide sequence covalently linked to a heterologous polypeptide sequence.


SEQUENCE LISTING (6 sequences)

SEQ ID NO: 1 - protein, 259 aa, Arabidopsis thaliana
MAEEYKNTVPEQETPKVATEESSAPEIKERGMFDFLKKKEEVKPQETTTLASEFEHKTQISEPESFVAKHEEEEHKPTLLEQLHQKHEEEEENKPSLLDKLHRSNSSSSSVSKKGEDGEKKKKEKKKKIVEGDHVKTVEEENQGVMDRIKEKFPLGEKPGGDDVPVVTTMPAPHSVEDHKPEEEEKKGFMDKIKEKLPGHSKKPEDSQVVNTTPLVETATPIADIPEEKKGFMDKIKEKLPGYHAKTTGEEEKKEKVSD

SEQ ID NO: 2 - DNA, 780 bp, Arabidopsis thaliana
  gi|42562192500-1279 Arabidopsis thaliana ERD10 (EARLY RESPONSIVE TO DEHYDRATION 10);actin binding (ERD10) mRNA, complete cds
atggcagaagagtacaagaacaccgttccagagcaggagacccctaaggttgcaacagaggaatcatcggcgccagagattaaggagcggggaatgttcgatttcttgaagaaaaaggaggaagttaaacctcaagaaacgacgactctcgcgtctgagtttgagcacaagactcagatctctgaaccagagtcgtttgtggccaagcacgaagaagaggaacataagcctactcttctcgagcagcttcaccagaagcacgaggaggaagaagaaaacaagccaagtctcctcgacaaactccaccgatccaacagctcttcttcctctgtaagtaaaaaaggtgaagacggtgagaagaagaagaaggagaaaaagaagaagattgttgaaggagatcatgtgaaaacagtggaagaagagaatcaaggagtaatggacaggattaaggagaagtttccactcggagagaaaccagggggtgatgatgtaccagtcgtcaccaccatgccagcaccacattcggtagaggatcacaaaccagaggaagaagagaagaaagggtttatggataagatcaaggagaagcttccaggccacagcaagaaaccagaggattcacaagtcgtcaacaccacaccgctggttgaaacagcaacaccgattgctgacatcccggaggagaagaagggatttatggacaagatcaaagagaagcttccaggttatcacgccaagaccactggagaggaagagaagaaagaaaaagtgtctgattaa

SEQ ID NO: 3 - protein, 185 aa, Arabidopsis thaliana [sequence omitted]

SEQ ID NO: 4 - DNA, 558 bp, Arabidopsis thaliana
atggctgaggaaatcaagaatgttcctgaacaggaggtgccaaaggtagcaacagaggaatcatcggcagaggttacagatcgtggattgttcgatttcttgggaaagaagaaagacgaaacaaaaccagaggagactccgatcgcttcagagtttgagcagaaggttcatatttcagagccggagccagaggttaaacacgaaagtcttcttgaaaagcttcaccgaagcgacagttcttctagctcctcaagtgaggaagaaggttcagatggtgagaagaggaagaagaagaaggagaagaagaagccaactactgaagttgaggtaaaggaggaagagaagaaagggtttatggagaagttgaaagagaagcttcctggacacaagaaacctgaagacggttcagccgtcgctgcggcaccggtggttgttcctcctcctgtggaagaagcgcatccagtggagaagaaagggattcttgagaagattaaggagaagcttccaggataccaccctaagaccaccgtagaggaggagaagaaagataaagaataa

SEQ ID NO: 5 - protein, 271 aa, Brassica napus
MAEEYKNASEEFKNVPEHESTPKVATTEEPSATTGEVKDRGLFDFLGKKEEVKPQETTTLESEFEHKAQVSEPPAFVAKHEEEEEREHKPTLLEKLHHKHEEEEEENKPSLLQKLHRSNSSSSSSDEEGEDGEKRKKEKKKIAEEDEKTKEDRKGVMEQIREKFPHGTKTEDDTPVIATLPVKEETVEHPEEKKRLMEKIKEKLPGHSEKPEDSQVVDTAAAVPVTEKTAEHPEEKKGLMGKIKEKLPGYHAKSTEEEEKKKEKESDDLEG

SEQ ID NO: 6 - DNA, 816 bp, Brassica napus
atggctgaagagtacaagaacgcttcggaggagttcaagaacgtccctgaacacgagtcgaccccaaaggttgccaccacggaggaaccatctgcgacgacgggagaggttaaggatcgtggactgtttgacttcttggggaaaaaagaggaagtgaaacctcaagagacgacgacacttgagtcagagtttgagcacaaggctcaggtctcggaaccgccggcgtttgtggcgaagcacgaagaagaggaagagagggagcataagcctactctcctcgagaagcttcaccataagcacgaggaagaagaagaagagaacaaacctagtctcctccagaagcttcaccgatccaatagctcttcctcttcaagcgatgaagaaggagaagatggtgagaagaggaagaaggagaagaagaagatcgctgaagaagatgagaaaacaaaggaagatagaaaaggggtaatggagcagatcagggagaagtttccacacggaacaaagacagaggatgacactccagtcatcgccaccctgccggtgaaggaggaaacggtagagcatccggaggagaagaaaagactgatggagaagatcaaggagaagcttccaggtcacagcgagaaaccagaggattctcaagtggtcgacacggcggctgcagtaccagtgacggagaaaacggcggagcatccggaagagaagaaaggactgatggggaagatcaaagagaagctcccaggttatcacgccaagagcactgaagaggaggagaagaagaaagaaaaggagtccgatgatttagaaggatga